(19) United States
(12) Patent Application Publication
(10) Pub. No.: US 2024/0335213 A1
(43) Pub. Date: Oct. 10, 2024

(54) ENDOSCOPIC INCISION DEVICES

(71) Applicant: HANGZHOU AGS MEDTECH CO., LTD., Hangzhou, Zhejiang (CN)

(72) Inventor: Baiming SHI, Hangzhou (CN)

(73) Assignee: HANGZHOU AGS MEDTECH CO., LTD., Hangzhou, Zhejiang (CN)

(21) Appl. No.: 18/292,913

(22) PCT Filed: May 27, 2023
(86) PCT No.: PCT/CN2022/095411

§ 371 Date: Jan. 28, 2024

(30) Foreign Application Priority Data
Jul. 28, 2021 (CN) 202110854782.5

(51) Int. Cl. A61B 17/3209; A61B 17/00; A61B 17/32

(52) U.S. Cl. CPC A61B 17/3209; A61B 17/320016; A61B 2017/00323; A61B 2017/00336; A61B 2017/00818; A61B 2017/32006

(57) ABSTRACT

The embodiments of the present disclosure provide an endoscopic incision device. The endoscopic incision device may include a sheath tube. The sheath tube may have a proximal end and a distal end, the sheath tube may be provided with a channel extending along an axial direction, and the channel may be able to accommodate a pulling portion. The pulling portion may include a pulling wire and a protruding portion disposed at a distal end of the pulling wire, and the pulling wire may move axially within the channel to cause the protruding portion to switch between a first state and a second state. When the protruding portion is in the first state, the protruding portion may be accommodated within the channel. When the protruding portion is in the second state, the protruding portion may protrude out of the distal end of the channel.

### CROSS-REFERENCE TO RELATED APPLICATIONS

This application claims priority to Chinese application No. 202110854782.5, filed on Jul. 28, 2021, the entire contents of each of which are incorporated herein by reference.

### TECHNICAL FIELD

The present disclosure relates to medical devices, in particular to endoscopic incision devices.

### BACKGROUND

Endoscopic retrograde cholangio pancreatography (ERCP) refers to a surgical procedure of inserting a guidewire or related instruments (e.g., an incision knife, a contrast catheter, etc.) into the common bile duct or pancreatic duct through the duodenal papilla (referred to as intubation). ERCP, with its unique advantages of minimal trauma and rapid recovery, is increasingly used in the diagnosis and treatment of biliary and pancreatic diseases. Intubation is a key technique in ERCP surgeries. However, the physiological structure of the duodenal papilla in some patients may be abnormal (for example, the folds of the duodenal papilla are thick and long), which increases the difficulty of intubation, reduces the success rate of intubation, and heightens the risk of postoperative complications such as pancreatitis and perforation

Therefore, the operation of reducing the difficulty of intubation, improving the success rate of intubation, and reducing the risk of postoperative complications is a concern in ERCP surgeries.

### SUMMARY

The embodiments of the present disclosure provide an endoscopic incision device. The endoscopic incision device includes a sheath tube. The sheath tube may have a proximal end and a distal end, the sheath tube may be provided with a channel extending along an axial direction, and the channel may be able to accommodate a pulling portion. The pulling portion may include a pulling wire and a protruding portion disposed at the distal end of the pulling wire, and the pulling wire may move axially out of the channel to cause the protruding portion to switch between a first state and a second state. When the protruding portion is in the first state, the protruding portion may be accommodated within the channel. When the protruding portion is in the second state, the protruding portion may protrude out of the distal end of the channel.

In some embodiments, when the protruding portion is in the second state, the protruding portion may form a joint portion with the distal end of the sheath tube, and a radial size of the joint portion may be greater than a diameter of the duodenal papilla. A radial size of the protruding portion may be a first size, and a radial size from a most distal end of the protruding portion in a first radial direction to a most distal end of the sheath tube in a second radial direction may be a second size. When the first size is greater than the second size, a radial size of the joint portion may be the first size. When the second size is greater than the first size, the radial size of the joint portion may be the second size.

In some embodiments, the radial size of the protruding portion in the second state may be larger than the radial size of the protruding portion in the first state. The radial size of the protruding portion in the first state may be less than or equal to a radial size of the distal end of the sheath tube. The radial size of the protruding portion in the second state may be greater than the radial size of the distal end of the sheath tube.

In some embodiments, the radial size of the protruding portion in the second state may be within a range of 2 to 10 mm. The radial size of the distal end of the sheath tube may be within a range of 1.8-2 mm.

In some embodiments, the channel includes a first channel configured to accommodate a guidewire and a second channel configured to accommodate the pulling portion.

In some embodiments, when switching from the first state to the second state, and the protruding portion may deflect away from an axis.

In some embodiments, the second channel is provided eccentrically.

In some embodiments, the distal end of the sheath tube may be provided with a cutting wire configured to cut tissue, and the second channel may collectively accommodate the pulling portion and the cutting wire.

In some embodiments, the distal end of the sheath tube may be provided with the cutting wire configured to cut the tissue, and the channel further includes a third channel, the second channel accommodates the pulling portion and the third channel configured to accommodate the cutting wire.

In some embodiments, the second channel or the third channel may be an injection channel.

In some embodiments, the channel further includes a fourth channel, the first channel may accommodate the guidewire, the second channel may accommodate the pulling portion, the third channel may accommodate the cutting wire, and the fourth channel may be an injection channel.

In some embodiments, a cross-section of the first channel may be a C-shaped cross-section, and the first channel may include a guidewire accommodating portion and a slit portion.

In some embodiments, the protruding portion may be composed of one or more pulling lines to form a semi-closed or closed loop.

In some embodiments, the protruding portion may be composed of one or more pulling lines to form a semi-closed or closed mesh.

In some embodiments, the protruding portion may be composed of one or more pulling lines to form a spiral.

In some embodiments, the radial size of the protruding portion may be approximately equal from the distal end to the proximal end, or gradually increase, or gradually decrease.

In some embodiments, the endoscopic incision device may further include a control portion, and the control portion may include a pulling handle. The pulling handle may push-pull axially or rotate to control the pulling wire to move axially within the channel, causing the protruding portion to switch between the first state and the second state.

In some embodiments, when the protruding portion is in the second state, the pulling handle may be further configured to control the pulling wire to move axially within the channel, causing the protruding portion to pull tissue.

In some embodiments, the distal end of the pulling wire and the proximal end of the protruding portion may be fixedly connected, or the distal end of the pulling wire and the proximal end of the protruding portion may be detachably connected.

In some embodiments, the protruding portion may be configured to pull duodenal papilla fold tissue.

One of the embodiments in the present disclosure provides a method for operating the endoscopic incision device of any one of the above embodiments. The method includes controlling the pulling wire to move axially from the proximal end to the distal end of the sheath tube within the channel of the sheath tube, causing the protruding portion to switch from the first state to the second state; and when the protruding portion is in the second state, controlling the pulling wire to move axially within the channel of the sheath tube, causing the protruding portion in the second state to pull the duodenal papilla fold tissue within an intubation channel.

In some embodiments, the method further includes controlling the pulling wire to move axially from the distal end to the proximal end of the sheath tube within the channel of the sheath tube, causing the protruding portion to switch from the second state to the first state; and when the protruding portion is in the second state, controlling the guidewire to extend from the distal end of the sheath tube and to enter common bile duct or pancreatic duct through the intubation channel.

One of the embodiments in the present disclosure provides an endoscope including the endoscopic incision device of any one of the above embodiments.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating a structure of an exemplary endoscopic incision device according to some embodiments of the present disclosure;

FIG. 2 is a schematic diagram illustrating a structure of another exemplary endoscopic incision device according to some embodiments of the present disclosure;

FIG. 3 is a schematic diagram illustrating different cross-sections of a sheath tube according to some embodiments of the present disclosure;

FIG. 4 is a schematic diagram illustrating different shapes of a protruding portion according to some embodiments of the present disclosure;

FIG. 5 is an enlarged schematic diagram illustrating M in FIG. 1;

FIG. 6 is an enlarged schematic diagram illustrating N in FIG. 2;

FIG. 7 is a schematic diagram illustrating an operation of an endoscopic incision device without a protruding portion according to some embodiments of the present disclosure;

FIG. 8 is a schematic diagram illustrating an operation of an endoscopic incision device with a pulling portion according to some embodiments of the present disclosure;

FIG. 9 is a schematic diagram illustrating the duodenal papilla before pulling according to some embodiments of the present disclosure;

FIG. 10 is a schematic diagram illustrating the duodenal papilla after pulling according to some embodiment of the present disclosure;

FIG. 11 is a flowchart illustrating a method for operating an endoscopic incision device according to some embodiments of the present disclosure;

FIG. 12 is a schematic diagram illustrating an endoscope according to some embodiment of the present disclosure.

### DETAILED DESCRIPTION

In order to illustrate the technical solutions related to the embodiments of the present disclosure, a brief introduction of the drawings referred to in the description of the embodiments is provided below. Obviously, the drawings described below are only some examples or embodiments of the present disclosure. Those skilled in the art, without further creative efforts, may apply the present disclosure to other similar scenarios according to these drawings. Unless apparent from the locale or otherwise stated, like reference numerals represent similar structures or operations throughout the several views of the drawings.

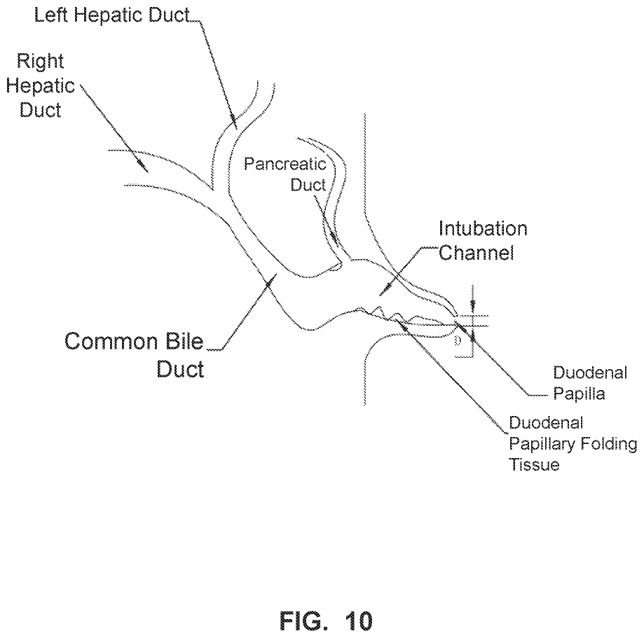

In ERCP procedures, when the duodenal papilla (referred to as papilla for short) folds of a patient are thick and long, the difficulty of the intubation is generally increased and the risk of intubation failure is encountered. Thick and long papilla folds generally refer to that there are many folds in an intubation channel (the intubation channel as shown in FIG. 10) in the papilla communicating a papilla opening with common bile duct and pancreatic duct, causing a shape of an inner wall of the intubation channel is in an S shape with a large fluctuation. These folds increase the resistance of a guidewire inserted into the intubation channel, prevent the guidewire from advancing in a normal direction, and prevent the guidewire from smoothly being inserted into the common bile duct or the pancreatic duct that the guidewire is expected to enter. In addition, the thick and long papillary folds may also affect an injection of a contrast agent, thereby affecting a developing effect of the common bile duct or pancreatic duct. In such cases, although successful intubation may be achieved by repeatedly adjusting intubation directions or using an operation of papillary preincision, this may cause damage to papillary tissue, increasing the risk of complications such as postoperative pancreatosis and perforation. It should be understood that the intubation not only includes the guidewire being inserted into the common bile duct or the pancreatic duct through the intubation channel, but also includes relevant instruments (for example, a balloon catheter, a net basket, an incision knife, a cell brush, a stent, a contrast catheter, and a snare) being inserted into the common bile duct or the pancreatic duct through the intubation channel under the guidance of the guidewire.

The embodiments of the present disclosure provide an endoscopic incision device, which may include a sheath tube, and the sheath tube has a proximal end and a distal end. The sheath tube may be provided with a channel extending along an axial direction, and the channel may be able to accommodate a pulling portion. The pulling portion includes a pulling wire and a protruding portion disposed at a distal end of the pulling wire. By moving the pulling wire axially in the channel, the protruding portion can be driven to be accommodated in the channel or protruded from of the distal end of the channel, and when the protruding portion is protruded from of the distal end of the channel, the protruding portion may be in contact with papillary folds to pull the papillary folds. The papillary folds are stretched and squeezed under the pulling action of the protruding portion and eventually approach a flattened state, so that the shape of the inner wall of the intubation channel is changed from an S-shape with a large undulation to a gentle S-shape, thereby making the intubation channel wider, and the guidewire is less likely to be blocked by the folds, which reduces the resistance of the guidewire being inserted into the intubation channel and allows the guidewire to be inserted into the common bile duct or the pancreatic duct that the guidewire is expected to enter, thereby reducing the difficulty of intubation, and increasing a success rate of intubation. It should be noted that the “distal end” and the “proximal end” involved in the embodiments of the present disclosure may respectively refer to one end that is far away from an operator and one end that is close to the operator when the intubation is performed.

The endoscopic incision device provided in the embodiments of the present disclosure would be described in detail below in connection with the accompanying drawings.

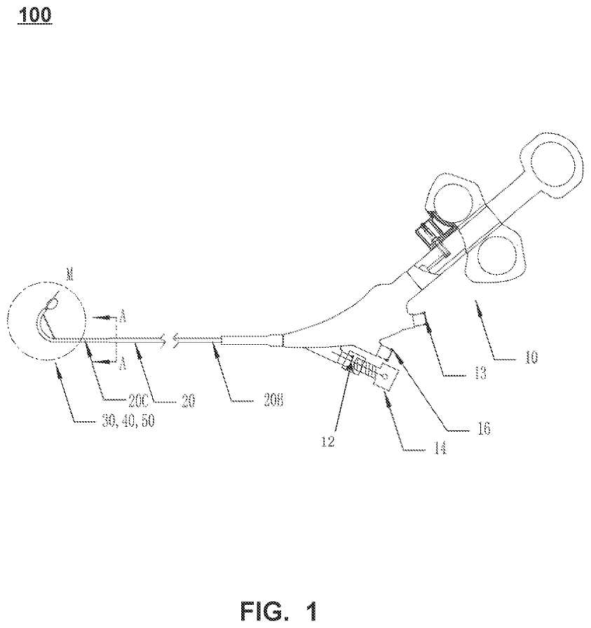

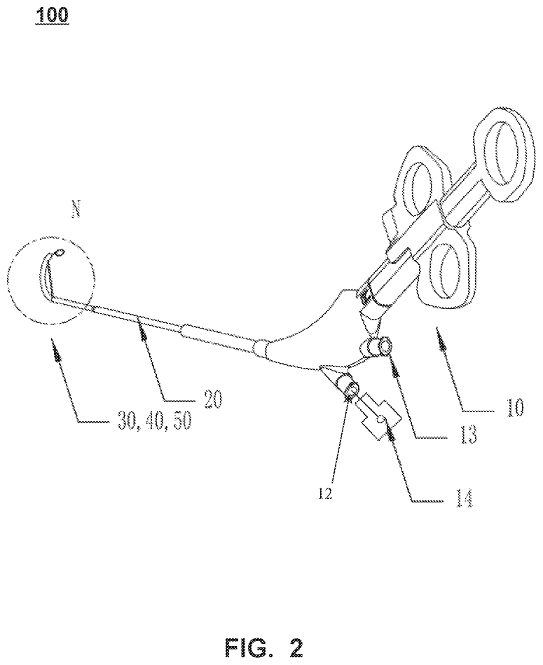

FIG. 1 is a schematic diagram illustrating a structure of an exemplary endoscopic incision device according to some embodiments of the present disclosure. FIG. 2 is a schematic diagram illustrating a structure of another exemplary endoscopic incision device according to some embodiments of the present disclosure.

As shown in FIG. 1 and FIG. 2, an endoscopic incision device 100 includes a sheath tube 20, and the sheath tube 20 has a proximal end and a distal end. The proximal end and the distal end of the sheath tube 20 may be respectively a right end and a left end of the sheath tube 20 illustrated in FIG. 1. In some embodiments, the sheath tube 20 is provided with a channel extending along an axial direction (or referred to as a length direction), and the channel may be configured to accommodate a pulling portion 40. The pulling portion 40 may include a pulling wire 42 and a protruding portion 41 disposed at a distal end of the pulling wire 42. The pulling wire 42 moves axially within the channel of the sheath tube 20 to cause the protruding portion 41 to switch between a first state and a second state. When the protruding portion 41 is in the first state, the protruding portion 41 is accommodated within the channel of the sheath tube 20. When the protruding portion 41 is in the second state, the protruding portion 41 protrudes out of the distal end of the channel of the sheath tube 20. The protruding portion 41 in the second state is capable of joining with the papillary folds for pulling duodenal papilla fold tissue (referred to as folds for short), the folds are stretched and squeezed under the pulling action, and gradually converge to a spreading state from an initial curved state, increasing a width of the intubation channel, reducing resistance of the guidewire in the channel, reducing the difficulty of intubation, and improving the success rate of intubation. More description of how the protruding portion 41 pulls the folds may be found elsewhere in the present disclosure, and would not be described in detail herein.

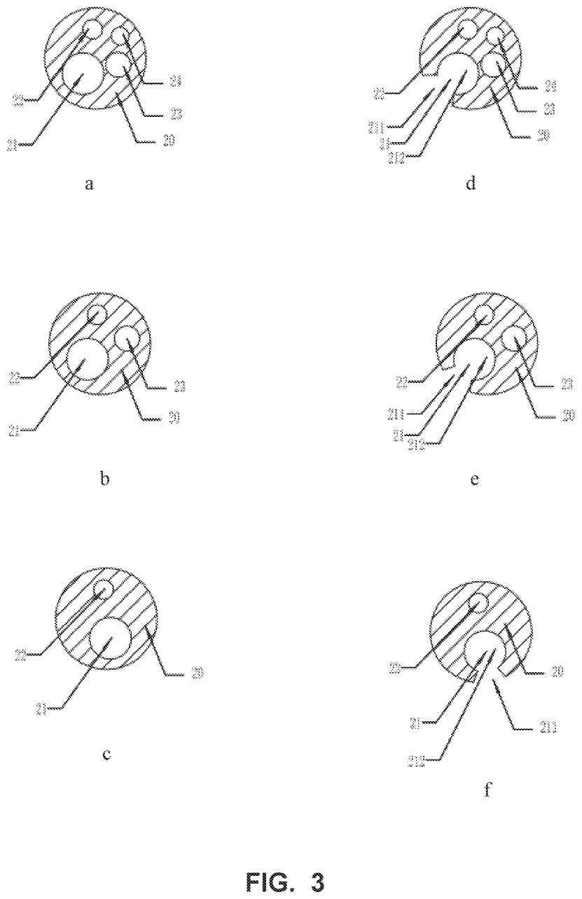

In some embodiments, as shown in FIG. 1 and FIG. 2, the endoscopic dissection device 100 may further include a control portion 10. The control portion 10 may include a pulling handle 14, the pulling handle 14 may control the pulling wire 42 to move axially in the channel (e.g., a second channel 22 shown in FIG. 3) in the sheath tube 20 by axially pushing-pulling or rotationally controlling the pulling wire 42, so that the protruding portion 41 switches between the first state and the second state. In some embodiments, when the protruding portion 41 is in the second state, the pulling handle 14 may also be configured to control the pulling wire 42 to move axially within the channel of the sheath tube 20 to cause the protruding portion 41 to pull the fold tissue. A proximal end of the protruding portion 41 may be connected to a distal end of the pulling wire 42 to cause the pulling wire 42 to move axially within the channel, thereby driving the protruding portion 41 to move, such that the protruding portion 41 may be moved from the inside of the channel to an outside of the distal end of the channel, and from the outside of the distal end of the channel to the inside of the channel. In some embodiments, the distal end of the pulling wire 42 and the proximal end of the protruding portion 41 are fixedly connected through welding, bonding, etc., and this connection is simple in process and solid in connection effect. In some embodiments, the distal end of the pulling wire 42 and the proximal end of the protruding portion 41 are detachably connected through snap, threaded connection, or interference fit. Merely by way of example, the distal end of the pulling wire 42 may be provided with external threads, and the proximal end of the protruding portion 41 may be provided with a threaded hole adapted to the external threads, so that the detachable connection between the pulling wire 42 and the protruding portion 41 may be realized by thread tightening and loosening. By detachably connecting the pulling wire 42 and the protruding portion 41, when the folds are normal and do not block intubation, the protruding portion 41 may be detached from the distal end of the pulling wire 42, and when the folds are abnormal (e.g., thick and long) and block the intubation, the protruding portion 41 is connected to the distal end of the pulling wire 42, and the problem of blocking intubation by the folds is solved by pulling the folds through the protruding portion 41, thereby realizing a flexible use of the endoscopic incision device. In some embodiments, the distal end of the pulling wire 42 may be directly constructed as the protruding portion 41, i.e., the pulling wire 42 is integrally formed with the protruding portion 41.

In some embodiments, as shown in FIG. 1, the control portion 10 is provided with an inlet 12 for the pulling portion 40 to enter into the channel of the sheath tube 20, and a proximal end of the pulling wire 42 protrudes out of the inlet 12 and is fixedly connected to the pulling handle 14. The inlet 12 may be provided with the internal threads. The pulling handle 14 may be provided with external threads adapted to the internal threads of the inlet 12. By threadedly assembling the pulling handle 14 with the inlet 12 and then rotating the pulling handle 14, the length of a portion of the pulling handle 14 located in the inlet 12 may be adjusted to drive the pull wire 42 to move axially in the channel.

In some embodiments, as shown in FIG. 2, the control portion 10 is provided with the inlet 12 for the pulling portion 40 to enter within the channel of the sheath tube 20, and a proximal end of the pulling wire 42 protrudes out of the inlet 12 and is fixedly connected to the pulling handle 14. The pulling handle 14 is installed inside the inlet 12. The operator may drive the pulling wire 42 axially within the channel by push-pulling the pulling handle 14 axially. Axial pushing-pulling of the pull handle 14 may be understood as pushing the pull handle 14 towards the inlet 12 or pulling the handle 14 outward from the inlet 12, and a direction of pushing and pulling may be an axial direction of the inlet.

In some embodiments, as shown in FIG. 1 and FIG. 2, the channel within the sheath tube 20 further accommodate a guidewire 50, and by inserting the guidewire 50 into common bile duct or pancreatic duct, a relevant instrument (for example, a balloon catheter, a net basket, an incision knife, a cell brush, a stent, a contrast catheter, a snare, etc.) may be inserted into common bile duct or pancreatic duct under the guidance of the guidewire 50, to perform a corresponding surgical operation. In some embodiments, the distal end of the sheath tube 20 may also be provided with a cutting wire 30 for cutting tissue, and the cutting wire may be accommodated in the channel in the sheath tube 20. In some embodiments, by applying high-frequency power to the cutting wire 30, cutting of the sphincter muscle, the common bile duct, or the distal end of the pancreatic duct may be achieved. In some embodiments, the channel in the sheath tube 20 may serve as an injection channel, and the injection channel may be used to allow a contrast agent to pass into the common bile duct or the pancreatic duct for visualization.

In some embodiments, the control portion 10 may be provided with a first opening 13, the first opening 13 may be connected to the channel (e.g., the injection channel) in the sheath tube 20 to cause the contrast agent to be injected into the injection channel through the first opening 13. In some embodiments, the control portion 10 may be provided with a second opening 16, and the second opening 16 may be connected to the channel (e.g., a first channel 21) in the sheath tube 20 to cause the guidewire 50 to be capable of entering the first channel 21 through the second opening 16.

The structure of the sheath tube 20 would be described in detail below in connection with the accompanying drawings.

FIG. 3 is a schematic diagram illustrating different cross-sections of a sheath tube according to some embodiments of the present disclosure.

In some embodiments, as shown in images (c) and (f) in FIG. 3, the channels in the sheath tube 20 may include a first channel 21 for accommodating the guidewire 50 and a second channel 22 for accommodating the pulling portion 40. The pulling wire 42 moves axially in the second channel 22 to cause the protruding portion 41 to switch between a first state and a second state. Specifically, when the protruding portion 41 is in the first state, the protruding portion 41 is accommodated in the second channel 22. When the protruding portion 41 is in the second state, the protruding portion 41 may protrude out of the distal end of the second channel 22. In some embodiments, the second channel 22 may also be an injection channel. In some embodiments, the second channel 22 may be configured to jointly accommodate the pulling portion 40 and the cutting wire 30.

In some embodiments, as shown in images (b) and (e) in FIG. 3, the channel in the sheath tube 20 may include a first channel 21, a second channel 22, and a third channel 23. The first channel 21 is configured to accommodate the guidewire 50, the second channel 22 is configured to accommodate the pulling portion 40, and the third channel is configured to accommodate the cutting wire 30. In some embodiments, either the second channel 22 or the second channel 23 may serve as the injection channel.

In some embodiments, as shown in images (a) and (d) in FIG. 3, the channel within the sheath tube 20 may include a first channel 21, a second channel 22, a third channel 23, and a fourth channel 24. The first channel 21 is configured to accommodate the guidewire 50, the second channel 22 is configured to accommodate the pulling portion 40, the third channel is configured to accommodate the cutting wire 30, and the fourth channel 24 serves as the injection channel.

In some embodiments, a cross-section of the first channel 21 may be in a closed shape. The closed shape may be understood as a shape with a closed contour. In some embodiments, as shown in images (a), (b), and (c) in FIG. 3, the cross-section of the first channel 21 may be circular. In some embodiments, the cross-section of the first channel 21 may also be a regular such as a triangle, a rectangle, a square pentagon, or the like, or an irregular shape with the closed contour.

In some embodiments, as shown in images (d), (e), and (f) in FIG. 3, the cross-section of the first channel 21 may be a C-shaped cross-section, i.e., a shape of a cross-section profile of the first channel 21 approximates a C-shape. Further, the first channel 21 may include a guidewire holding portion 212 and a slit portion 211. As shown in FIG. 1, the sheath tube 20 may include a distal port 20A disposed at a distal end thereof, a proximal port 20B disposed at a proximal end thereof, and an intermediate port 20C disposed between the distal end 20A and the proximal end 20B. In some embodiments, the first channel 21 may be disposed within the wall of the sheath tube 20, extending along the distal end to the proximal end of the sheath tube 20. In some embodiments, the slit portion 211 is disposed in a portion of the first channel 21 between the proximal port 20B and the intermediate port 20C. In some embodiments, the slit portion 211 may be an axial opening of the sheath tube 20 extending between the proximal port 20B and the intermediate port 20C. In some embodiments, the slit portion 211 may be a weakened region of the sheath tube 20, and a radial opening region is a slit extending between the proximal port 20B and the intermediate port 20C. In some embodiments, when the guidewire 50 is accommodated within the first channel 21, a distal end of the guidewire 50 may be inserted within the intermediate port 20C, passing through the first channel 21, and exposing from the distal end 20A of the sheath tube 20. By using the intermediate port 20C as a guidewire inlet to place the guidewire 50 in the first channel 21, it may not individually provide the guidewire inlet (e.g., the second opening 16) on the control portion 10. At the same time, through the design of the slit portion 211 of the first channel 21 and the distal end of the guidewire 50 being inserted into the intermediate port 20C, the guidewire 50 may be easily and quickly separated from the sheath 20 or the guidewire 50 may be accommodated in the first channel 21, thereby realizing fast switching technology. Merely by way of example, after the guidewire 50 is inserted into the common bile duct or the pancreatic duct using the endoscopic dissection device 100, the arrangement of the slit portion 211 may facilitate quick disengagement of the guidewire 50 from the sheath tube 20, causing the guidewire 50 to be retained inside the common bile duct or the pancreatic duct, while the sheath tube 20 and the corresponding instruments are withdrawn from a body of a patient, so that the sheath tube 20 of an instrument for performing other surgical operations may be inserted into the common bile duct or the pancreatic duct under the guidance of the guidewire 50, to achieve the purpose of quickly switching instruments. In some embodiments, a width of the slit portion 211 may be smaller than a diameter of the guidewire 50, which ensures that the guidewire 50 does not detach from the sheath tube 20 through the slit portion 211 without an external force, thereby reducing the risk that the guidewire 50 is separated from the sheath 20 during a process of guiding the instrument to insert into the common bile duct or the pancreatic duct.

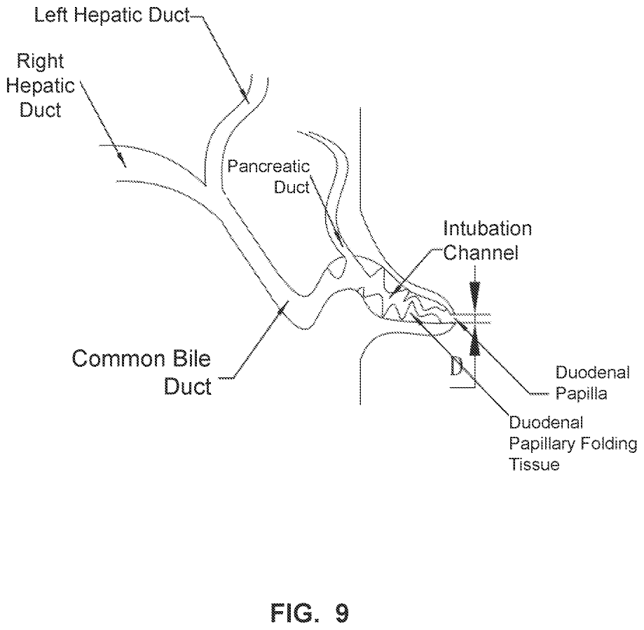

In the endoscopic incision device provided in the embodiments of the present disclosure, the protruding portion is switched from the first state to the second state, the protruding portion 41 protrudes out of the distal end of the second channel 22 and undergoes radial elastic opening and/or deflection, so that a radial size of the protrusion portion 41 or a radial size of the joint portion formed by the protrusion portion 41 and the distal end of the sheath pipe 20 is increased to be larger than a diameter of the duodenal papilla (for example, a diameter D of the duodenal papilla shown in FIG. 9), so that the protrusion portion 41 may be well joint (or fitted) with the folds. In some embodiments, when being in the second state, the protruding portion 41 is driven by the pulling wire 42 to move along an axial direction of the intubation channel, the protruding portion 41 may pull the folds to stretch and squeeze the folds, which is equivalent to smoothing the folds, causing the folds to be converged to a spreading state from a curved state, increasing the width of the intubation channel, thereby reducing the difficulty of intubation, and improving a success rate of intubation. In some embodiments, during a process of switching from the first state to the second state, the protruding portion 41 may also have a certain pulling effect on the folds. The protruding portion 41 and how the protruding portion 41 pulls the folds would be described in detail below in connection with the accompanying drawings.

In some embodiments, to cause the protruding portion 41 to be able to undergo the radial elastically opening and/or deflection when switching from the first state to the second state and the protruding portion 41 to return to the shape the protruding portion 41 in the first state when switching from the second state to the first state, the protruding portion 41 needs to have a certain elastic deformation capability. Therefore, a material of the protruding portion 41 may be a shape memory material having a certain elasticity. In some embodiments, the material of the protruding portion 41 may include but is not limited to, a metal material such as stainless steel, a nickel-titanium alloy, an iron-platinum alloy, etc., a polymer material such as polyurethane, polyolefin, epoxy resin, etc., a shape memory ceramic material, etc., or a combination thereof.

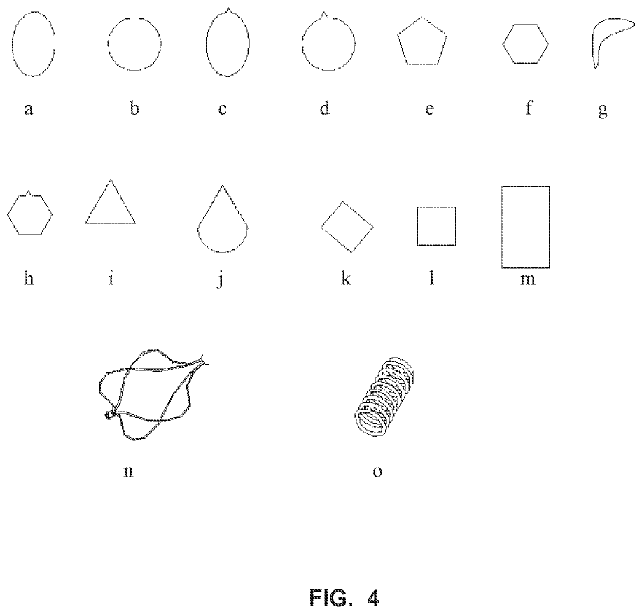

In some embodiments, the protruding portion 41 may include one or more pulling lines, forming a semi-closed or closed loop. The semi-closed loop may be understood as a loop that is not fully closed and has an opening. In some embodiments, the pulling line may be identical to the pulling wire 42, i.e., the pulling line and the pulling wire 42 have the same diameter, material, etc. In some embodiments, the protruding portion 41 may be a portion of the pulling wire 42. For example, a portion of the distal end of the pulling wire 42 may be bent to form a semi-closed or closed loop. In some embodiments, the protruding portion 41 may be composed of one or more pulling lines to form a semi-closed or closed loop, and then be fixedly or detachably connected to the distal end of the pulling wire 42. In some embodiments, the protruding portion 41 may be pre-shaped as a ring-shaped of variable radial size. Specifically, the protruding portion 41 may have different radial sizes when it is not under force or under different degrees of force. Merely by way of example, when the protruding portion 41 is in the first state, the protruding portion 41 is accommodated in the second channel 22, the second channel 22 may generate a restrain force on the radial expansion of the protruding portion 41, causing a radial size of the protruding portion 41 to be smaller. When the protruding portion 41 is in the second state, the protruding portion 41 is no longer subjected to force, and the protruding portion 41 undergoes radial expansion and the radial size of the protruding portion 41 becomes larger. In some embodiments, the protruding portion 41 may be a circular or non-circular annular structure (e.g., regular or irregular shape such as triangular, rectangular, pentagonal, hexagonal). Merely by way of example, the protruding portion 41 may include but is not limited to, an annular structure having a shape such as shown in images (a)˜(m) in FIG. 4. The protruding portion 41 of the annular structure in different shapes may cause a radial size change rate of the protruding portion 41 from the first state to the second state to be different, and also cause a direction of force exerted by the protruding portion 41 on the folds to be different, so as to be suitable for the intubation of the intubation channel having folds of different curvature degrees.

In some embodiments, the protruding portion 41 may be composed of one or more pulling lines of a shape memory material having a certain degree of elasticity, forming the semi-closed or closed mesh. Merely by way of example, the protruding portion 41 may be mesh-shaped as shown in image (n) in FIG. 4. By setting the protruding portion 41 in the mesh-shaped, when switching from the first state to the second state, the protruding portion 41 may undergo radial elastic expansion and/or deflection in a plurality of directions according to the specific conditions of the folds (e.g., distribution of the folds in the intubation channel, shapes of the folds, the curvature degree of the folds, etc.), so that a joint area of the protruding portion 41 in the second state with the folds is larger, and when the protruding portion 41 in the second state is driven by the pulling wire to pull the folds, the protruding portion 41 may have a better pulling effect on the folds. Specifically, the larger the joint area of the protruding portion 41 and the folds, the larger the area of the folds that the protruding portion 41 can be pulled and squeezed when moving axially, which not only causes the spreading of the folds to have a better effect, but also reduces time for making the folds tend to converge from the curved state to the spreading state, thereby increasing the efficiency of the intubation.

In some embodiments, the protruding portion 41 may be composed of one or more pulling wires to form a spiral. Merely by way of example, the protruding portion 41 may be in a spiral shape as shown in image (0) in FIG. 4. By setting the protruding portion 41 in the spiral shape, when switching from the first state to the second state, the protruding portion 41 may undergo radial elastic expansion and/or deflection in the plurality of directions according to the specific conditions of the folds (e.g., the distribution of the folds in the intubation channel, the shapes of the folds, the curvature degree of the folds, etc.), so that the joint area of the protruding portion 41 in the second state with the folds is larger, and when the protruding portion in the second state is driven by the pulling wire 42 to pull the folds, the protruding portion 41 may have the better pulling effect on the folds. In addition, when a spiral-shaped protruding portion 41 is in the second state and in combination with the folds, the force generated by the protruding portion 41 on the folds that is conducted and propelled along the axial direction is more concentrated, causing the protruding portion 41 to have the better pulling effect on the folds. Specifically, the more concentrated the force generated by the protruding portion 41 on the folds that is conducted and propelled along the axial direction, the greater the deformation degree of the folds by stretching and squeezing under the pulling of the protruding portion 41, so that the folds may converge from the curved state to the flattened state in a faster and better manner, thereby facilitating improving the efficiency and the success rate of the intubation.

In some embodiments, the radial sizes of the spiral-shaped protruding portion 41 may be substantially equal from the distal end to the proximal end. In some embodiments, the radial sizes of the protruding portion 41 being substantially equal from the distal end to the proximal end may refer to that a change rate of the radial sizes of the protruding portion from the distal end to the proximal is within 5%. In some embodiments, the radial sizes of the protruding portion 41 being substantially equal from the distal end to the proximal end may refer to that the change rate of the radial sizes of the protruding portion from the distal end to the proximal end is within 3%. In some embodiments, the radial sizes of the protruding portion 41 being substantially equal from the distal end to the proximal end may refer to that a change rate of the radial sizes of the protruding portion from the distal end to the proximal is within 1%. By making the radial sizes of the spiral-shaped protruding portions 41 substantially equal from the distal end to the proximal end, when the protruding portions 41 are joined with the folds, the pulling effect on the folds is relatively uniform, which is suitable for the protruding portion to pulling of the folds stably.

In some embodiments, the radial sizes of the spiral-shaped protruding portion 41 may be different from the distal end to the proximal end, and different radial sizes from the distal end to the proximal end may suit different fold structures. In some embodiments, the radial sizes of the spiral-shaped protruding portion 41 may gradually increase from the distal end to the proximal end. When the spiral-shaped protruding portion 41 with the radial sizes gradually increasing from the distal end to the proximal end moves axially, initially the joint area of the protruding portion with the folds is small, and as the spiral-shaped protruding portion 41 continues to move axially, the joint area of the protruding portion 41 with the folds grows larger and larger, the pulling effect on the folds is gradually increased, which is suitable for a pulling situation of a region in which blockage of the folds is relatively concentrated in the intubation channel. In addition, the spiral-shaped protruding portion 41 with the radial sizes that gradually increase from the distal end to the proximal end may be quickly accommodated in the channel of the sheath tube 20 (e.g., the second channel 22) or protruded out of the channel of the sheath tube 20 to achieve a quick switch between the first state and the second state of the protruding portion 41 without affecting subsequent operations of the guidewire entering the intubation channel after the folds have been pulled by the spiral-shaped protruding portion 41. In some embodiments, the radial sizes of the spiral-shaped protruding portion 41 may also be gradually reduced from the distal end to the proximal end.

In some embodiments, the structure of the protruding portion 41 may be designed based on a principle of umbrella opening and closing to achieve the purpose that the protruding portion 41 switches from the first state to the second state and when the protruding portion is in the second state, the radial size of the protruding portion 41 increases and is larger than the diameter of the duodenal papilla. Specifically, the protruding portion 41 may include a rod member (similar to an umbrella handle) and a body portion (similar to an umbrella surface) connected to a distal end of the rod member. The rod member is sleeved with a sliding member capable of sliding axially relative to the rod member, and the sliding member is connected to the body portion through a plurality of connecting members (similar to umbrella bones), so that when the sliding member slides toward the distal end of the rod member, the sliding member is able to drive the body portion to open through the plurality of connecting members, and when the sliding member slides toward a proximal end of the rod member, the sliding member is able to drive the body portion to close through the plurality of connecting members. In some embodiments, the body portion may be a mesh structure as shown in image (n) in FIG. 4, and sliding of the sliding member over the rod member may drive the mesh structure to open or close. In some embodiments, the proximal end of the rod member may be connected to the distal end of the pulling wire 42, and the interior of the channel of the sheath tube 20 may be provided with one or more additional pulling wires for driving the sliding member to slide on the rod member. When the protruding portion 41 protrudes out of the distal end of the channel, the sliding of the sliding member on the rod may be driven by artificially pushing-pulling the one or more additional pulling wires axially within the channel of the sheath tube 20, thereby driving the body portion to open or close. In some embodiments, the one or more additional pulling wires in the above embodiments may be replaced with a telescoping rod, and the sliding member may be driven to slide on the rod using the telescoping movement of the telescoping rod. In some embodiments, by controlling an axial movement distance of the additional pulling wires in the channel or controlling an elongation length of the telescopic rod, the body portion may have different opening degrees, so that, the protruding portion 41 may have different radial sizes when being in the second state, and thereby being suitable for pulling the folds in the intubation channel having different diameters or for pulling the folds having different shapes, curvature degrees, etc., ensuring that the protruding portion 41 has a large joint area with the folds, so that the protruding portion 41 has the better pulling effect on the folds. By designing the protruding portion 41 based on the principle of the umbrella, not only the radial size of the protruding portion 41 in the second state may be increased to realize the better pulling effect on the folds, but also the switching efficiency of the protruding portion 41 from the first state to the second state may be improved.

In some embodiments, the protruding portion 41 may be a balloon, and accordingly, the pulling wire 42 may be replaced with a pulling catheter in communication with the balloon. When the protruding portion 41 is in the first state, the pressure within the balloon is small and the balloon has a relatively small size and is accommodated within the channel of the sheath tube 20, and as the pulling catheter moves axially toward the distal end, the protruding portion 41 is in the second state, where the balloon protrudes out of the distal end of the channel of the sheath tube 20, and at this time, gas is communicated through the pulling catheter into the balloon, which causes the balloon to undergo radial expansion, and the radial size of the balloon is increased and can be well integrated with the folds to achieve pulling of the folds. In some embodiments, when the protruding portion 41 is in the second state, the radial size of the balloon may be controlled by the volume of the gas passed into the balloon, such that the protruding portion 41 is suitable to pull the folds in the intubation channel having different diameters or to pull the folds of different shapes, curvature degrees, etc., thereby ensuring that the protruding portion 41 has a relative great joint area with the folds, and making the protruding portion 41 to have the better pulling on the folds.

In some embodiments, since the folds may be distributed at any position in a circumferential direction of an inner wall of the intubation channel, in order to that the protruding portion 41 has a uniform pulling effect on the folds in the circumferential direction of the inner wall of the intubation channel to stably pull the folds, the protruding portion 41 may rotate (e.g., rotate around a center axis of the pulling wire 42) simultaneously during a process of moving axially to achieve the pulling of the folds, such that the folds in the circumferential direction of the inner wall of the intubation channel may join with the protruding portion 41. In some embodiments, an axial rotation of the protruding portion 41 may be achieved by rotating the sheath tube 20. In some embodiments, the axial rotation of the protruding portion 41 may be achieved by rotating the pulling wire 42. In some embodiments, a rotation mechanism may be provided between the protruding portion 41 and the distal end of the pulling wire 42, which may drive the protruding portion 41 to rotate axially without the pulling wire 42 rotating. In some embodiments, the rotation mechanism may be a driving mechanism (e.g., a motor) capable of outputting a rotational movement, and driving the protruding portion 41 to perform the axial rotation through the driving mechanism, and the rotation mechanism may eliminate the need to artificially rotate the protruding portion 41 in the axial direction, which is conducive to improving the efficiency and effectiveness of the protruding portion 41 pulling the folds.

In some embodiments, friction between the protruding portion 41 and the folds may be increased by increasing surface roughness of the protruding portion 41 to prevent the protruding portion 41 from slipping out during pulling the folds and not being able to continue pulling the folds. In some embodiments, the surface roughness of the protruding portion 41 may be increased by providing a plurality of fine structures (e.g., bumps, pits, etc.) on a surface of the protruding portion 41.

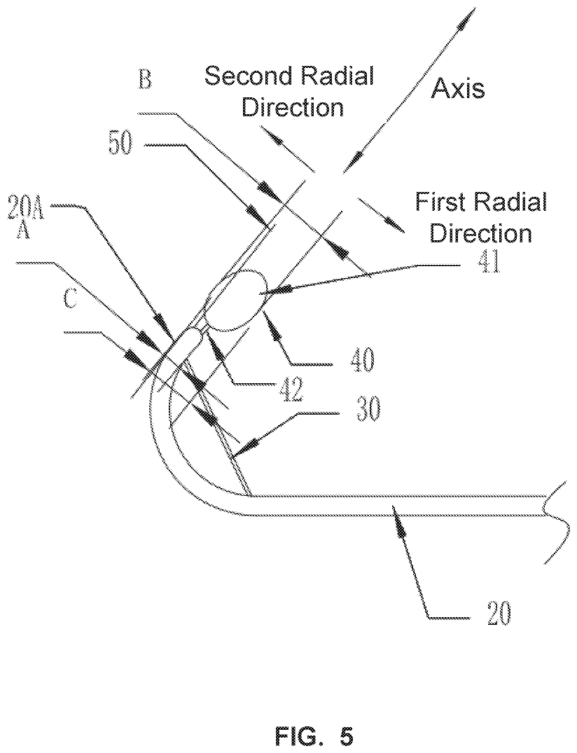

In some embodiments, when the protruding portion 41 switches from the first state to the second state, the protruding portion 41 may be elastically opened to facilitate joining with the folds and pulling the folds. Specifically, as an example, the protruding portion 41 is in a ring-shape, as shown in FIG. 5, when being in the first state, the ring-shaped protruding portion 41 may be pre-accommodated in the channel (i.e., the second channel 22) of the sheath tube 20, at this time, the radial size of the protruding portion 41 is less than or equal to the radial size of the distal end of the sheath tube 20. In some embodiments, the radial size of the protruding portion 41 in the first state is less than a diameter of the second channel 22. By axially pushing or rotating the pulling handle 14 to drive the pulling wire 42 to move axially from the proximal end to the distal end within the second channel 22, the protruding portion 41 may be switched from the first state to the second state. When being in the second state, the ring-shaped protruding portion 41 may protrude out of the distal end of the second channel 22 and undergo elastic opening, and accordingly, the radial size of the protruding portion 41 increases, i.e., a radial size B (or referred to as a first size) of the protruding portion 41 in the second state is larger than the radial size of the protruding portion 41 in the first state, and the radial size B of the protruding portion 41 in the second state is larger than the radial size of the distal end of the sheath tube 20. Further, the radial size of the protruding portion 41 in the second state may be larger than the diameter of the duodenal papilla (i.e., a diameter D of the duodenal papilla shown in FIG. 9 or FIG. 10). In some embodiments, the radial size of the distal end of the sheath tube 20 may be within a range of 1.8-2 mm. In some embodiments, the radial size of the protruding portion 41 in the second state may be within a range of 2-10 mm. In some embodiments, the radial size of the protruding portion 41 in the second state may be within a range of 4-10 mm. In some embodiments, the radial size of the protruding portion 41 in the second state may be within a range of 6-10 mm. In some embodiments, the radial size of the protruding portion 41 in the second state may be within a range of 8-10 mm. Through the above settings, the protruding portion 41 that is in the second state and undergoes the elastic opening may join with duodenal papilla fold tissue and pull the duodenal papilla fold tissue, such that the intubation channel is wider, the resistance of the fold tissue encountered by the guidewire when it enters the intubation channel becomes smaller, thereby decreasing the difficulty of the intubation and improving the success rate of the intubation.

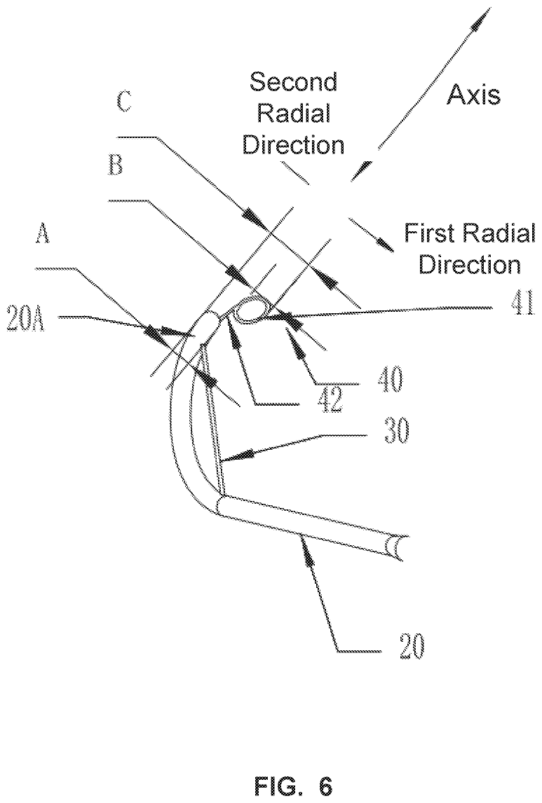

In some embodiments, when the protruding portion 41 switches from the first state to the second state, the protruding portion 41 may be deflected away from the axis (e.g., as shown in the axis in FIG. 5 or FIG. 6) to facilitate joining with the folds and pulling the folds. In some embodiments, the second channel 22 may be provided eccentrically (i.e., an axis of the second channel 22 does not coincide with an axis of the sheath tube 20) to increase the deflection angle of the protruding portion 41 in the second state away from the axis, which is beneficial to the combination of the protruding portion 41 and the duodenal papilla fold tissue, thereby having the better pulling effect. In some embodiments, as an example, the protruding portion 41 is in ring-shape, as shown in FIG. 6, the ring-shaped protruding portion 41 may be pre-accommodated into the sheath tube 20 channel (i.e., the second channel 22) when being in the first state, and at this time, the radial size of the protruding portion 41 is smaller than the diameter of the second channel 22. When the pulling wire 42 is driven to axially move from the proximal end to the distal end in the second channel 22 by the axial pushing or rotation of the pulling handle 14, the protruding portion 41 may be switched from the first state to the second state. When being in the second state, the ring-shaped protruding portion 41 may be protruding out of the distal end of the second channel 22, at this time the ring-shaped protruding portion 41 is deflected away from the axis. A radial size C (a second size) from a first most distal end of the ring-shaped protruding portion 41 in a first radial direction to a second most distal end of the distal end 20A of the sheath tube in a second radial direction is larger than a diameter of the duodenal papilla fold tissue, which causes the deflected protruding portion 41 in the second state to join with the duodenal papilla fold tissue to pull the duodenal papilla fold tissue, so that the intubation channel is wider, the resistance encountered by the guidewire when it enters the intubation channel becomes smaller, thereby reducing the difficulty of intubation and improves the success rate of intubation.

In some embodiments, when the ring-shaped protruding portion 41 switches from the first state to the second state, the protruding portion 41 may be elastically opened and deflected away from the axis to facilitate joining with the folds and pulling the folds. The radial size B (the first size) of the protruding portion 41 in the second state may be larger than the radial size of the protruding portion 41 in the first state. Further, when the protruding portion 41 is in the second state, the protruding portion 41 and the distal end of the sheath tube 20 may form a joint portion, and the radial size of the joint portion may be larger than the diameter of the duodenal papilla fold tissue, so that combination between the protruding portion 41 and the duodenal papilla fold tissue may pull the duodenal papilla fold tissue by relying on the double action of “the elastic opening the of the protruding portion” and the “deflection of the protruding portion, and accordingly, the pulling effect is better, the intubation channel is wider, and the resistance of the fold tissue encountered by the guidewire when it enters the intubation channel is smaller, thereby reducing the difficulty of intubation and improving the success rate of intubation. In some embodiments, when the radial size B (the first size) of the protruding portion 41 in the second state is larger than the radial size C (the second size) from the first most distal end of the protruding portion 41 in the first radial direction to the second most distal end of the distal end of the sheath tube in the second radial direction, the first size may be used as a radial size of the joint portion. When the radial size B (first size) of the protruding portion 41 in the second state is smaller than the radial size C (second size) from the first most distal end of the protruding portion 41 in the first radial direction to the second most distal end of the distal end of the sheath tube in the second radial direction, the second size may be used as the radial size of the joint portion.

In some embodiments, the channel of the sheath tube 20 may include a negative pressure channel (not shown in the drawings), and by generating negative pressure within the channel, the folds within the intubation channel may be made to be pulled taut under an action of the negative pressure suction, and width of the intubation channel may be made to be enlarged, and the resistance of fold tissue encountered by the guidewire when it enters the intubation channel becomes smaller, thereby reducing the difficulty of the intubation, and improving the success rate of the intubation.

The operation process of the endoscopic incision device 100 provided by the embodiments of the present disclosure would be described in detail below in connection with the accompanying drawings.

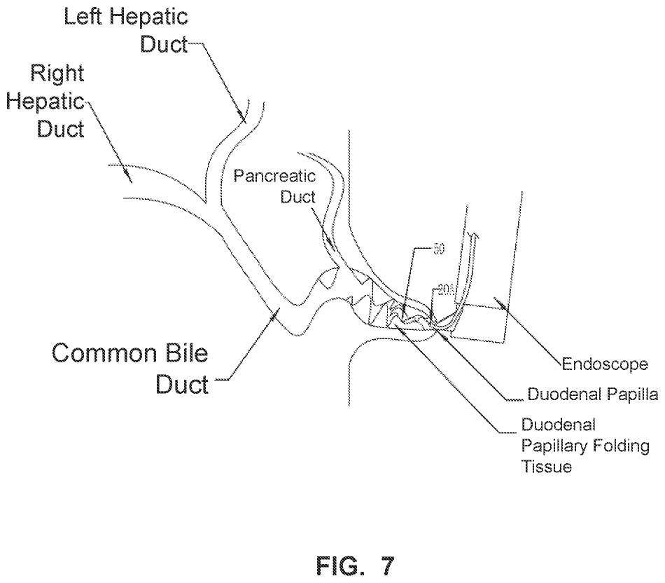

When using the endoscopic incision device 100, the operator may enter the body through an endoscope to find an opening of the duodenal papilla, and by adjusting a bending angle and a bending direction at the distal end 20A of the sheath tube 20 of the endoscopic incision device 100, the distal end 20A of the sheath tube 20 may be made to enter the opening of the duodenal papilla. In some embodiments, as shown in FIG. 7, when the sheath tube 20 that is not provided with the pulling portion 40 enters the duodenal papilla fold tissue, because the duodenal papilla fold tissue has a certain length in a radial direction of the intubation channel, when the guidewire 50 enters a path of the common bile duct from the opening of the duodenal papilla, the resistance of the guidewire 50 joining with the fold tissue is relatively great, causing a poor passageability of the guidewire 50. In addition, the thick and long folds also affect diffusion of injection of contrast agent, making the cholangiographic effect poorer and unable to accurately locate a position of the common bile duct or the pancreatic duct, thereby causing a greater obstacle to the guidewire 50 subsequently entering the common bile duct or the pancreatic duct.

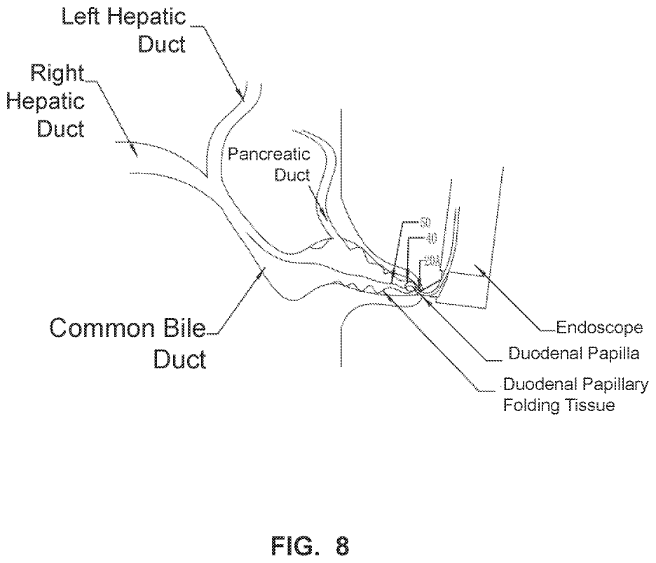

In some embodiments, as shown in FIG. 8, after the sheath tube 20 of the endoscopic incision device 100 enters the duodenal papilla (the diameter D of the duodenal papilla is larger than the radial size A of the distal end of the sheath tube 20 shown in FIG. 6), the control portion 10 may drive the pulling wire 42 to move from the proximal end to the distal end within the second channel 22, and the protruding portion 41 connected to the distal end of the pulling wire 42 protrudes out of the sheath tube from the sheath tube (the first state switches to the second state), the radial size of the protruding portion 41 becomes larger, and the radial size B of the protruding portion 41 in the second state is larger than the radial size A of the distal end of the sheath tube, and the protruding portion 41 protruding out of the distal end 20A of the sheath tube is composed of the shape memory material, which maintains a certain degree of rigidity in the state of being not subjected to force. The protruding portion 41 in the second state is in contact with the duodenal papilla fold tissue to be joined, and is driven by the pulling wire 42 to move axially within the channel and stretch and squeeze the duodenal papilla fold tissue through a pulling action, and the folds is flattened by the folds after being pulled, and the intubation channel from an original duodenal papillary to the common bile duct or the pancreatic duct is changed from a steep S-shape shown in FIG. 9 to a gentle S-shape shown in FIG. 10. When the protruding portion 41 completes the pulling action on the duodenal papilla fold tissue, the control portion 10 may drive the pulling wire 42 to move from distal end to proximal end within the second channel 22, and the protruding portion 41 connected to the distal end of the pulling wire 42 is accommodated from the exterior of the sheath tube 20 to the interior of the second channel 22 of the sheath tube 20 (the second state is switched to the first state), at which time the guidewire 50 extends out from the distal end 20A of the sheath tube 20, and the intubation channel from the duodenal papilla to the common bile duct after being pulled is more suitable for the diffusion of the contrast agent and makes the contrast effect better, and is also more suitable for the guidewire 50 to enter the common bile duct, thereby improving the success rate of intubation.

In some embodiments, the protruding portion 41 is deflected away from the axis when the protruding portion 41 is switched from the first state to the second state, and the pulling portion of the duodenal papilla fold tissue is completed by a deflected protruding portion 41, and the same effect as described above may be achieved. In some embodiments, the second channel 22 accommodating the protruding portion 41 is disposed eccentrically, causing a deflection angle at which the protruding portion 41 is deflected away from the axis when the protruding portion 41 is switched from the first state to the second state is larger, which is more conducive to joining with the duodenal papilla fold tissue to have the better pulling effect.

The embodiments of the present disclosure also provide a method for operating an endoscopic incision device, which may be applied to the endoscopic incision device 100 in any embodiment of the present disclosure.

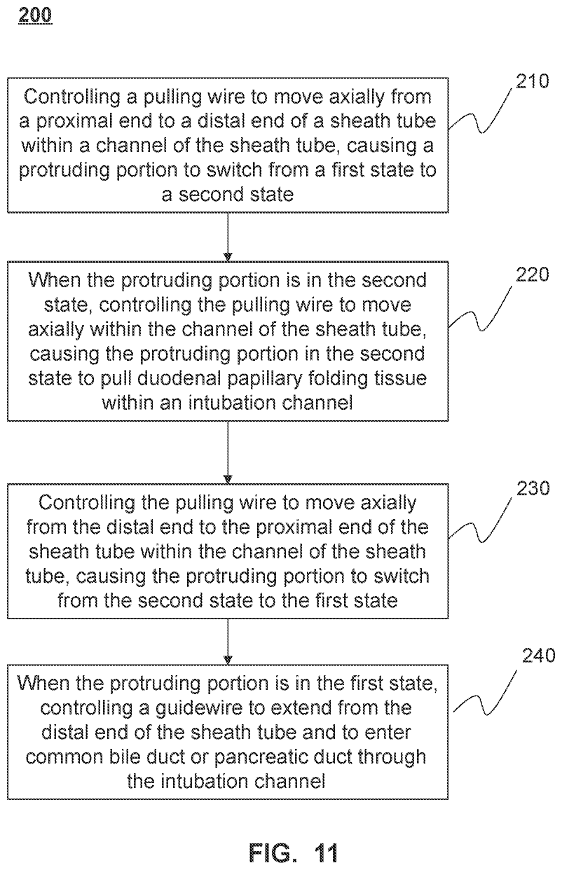

FIG. 11 is a flowchart illustrating a method for operating an endoscopic incision device according to some embodiments of the present disclosure.

As shown in FIG. 11, a method for operating the endoscopic incision device 200 may include the following operations.

In 210, a pulling wire 42 is controlled to move axially from the proximal end to the distal end of a sheath tube 20 within a channel of the sheath tube 20, causing a protruding portion 41 to switch from a first state to a second state. Specifically, an operator may axially push-pull or rotationally control, through a control handle 14 in a control portion 10, a pulling wire 42 to move axially from the proximal end to the distal end within the channel (e.g., a second channel 22) of the sheath tube 20 to drive the protruding portion 41 connected to the distal end of the pulling wire 42 to move from being accommodated within the channel (i.e., a first state) to being protruded out of the distal end of the channel (i.e., a second state).

In 220, when the protruding portion 41 is in the second state, the pulling wire 42 is controlled to move axially within the channel of the sheath tube 20, causing the protruding portion 41 in the second state to pull duodenal papilla fold tissue within an intubation channel. Specifically, when the protruding portion 41 is in the second state, the protruding portion 41 is within the intubation channel. When the protruding portion 41 switches from the first state to the second state, a radial expansion and/or deflection of the protruding portion 41 occurs, causing the protruding portion 41 in the second state to be better integrated with the duodenal papilla fold tissue within the intubation channel. The operator may axially push-pull or rotationally control, through the control handle 14 in the control portion 10, the pulling wire 42 within the channel (e.g., the second channel 22) of the sheath tube 20 to axially move from the proximal end to the distal end and axially move from the distal end to the proximal end to drive the protruding portion 41 to axially move back and forth within the intubation channel. During the back-and-forth axial movement of the protruding portion 41, the protruding portion 41 has a pulling effect on the duodenal papilla fold tissue, so that the duodenal papilla fold tissue may be converged to a spreading state from the curved state, to increase the width of the intubation channel.

In 230, the pulling wire 42 is controlled to move axially from the distal end to the proximal end of the sheath tube 20 within the channel of the sheath tube 20, causing the protruding portion 41 to switch from the second state to the first state. Specifically, after the protruding portion 41 completes pulling the duodenal papilla fold tissue to increase the width of the intubation channel, the operator may axially push-pull or rotationally control, the control handle 14 in the control portion 10, the pulling wire 42 to move axially from the distal end to the proximal end within the channel of the sheath tube 20 to drive the protruding portion 41 to move from being protruded out of the distal end of the sheath tube 20 (the second state) to being accommodated inside the sheath tube 20 (the first state), which facilitates performing subsequent operations, for example, an insertion of a guidewire or an injection of a contrast agent through the intubation channel, et.

In 240, when the protruding portion is in the first state, the guidewire is controlled to extend from a distal end 20 A of the sheath tube 20 and enter the common bile duct or the pancreatic duct through the intubation channel. Specifically, the operator may first place the guidewire 50 into a channel (e.g., the first channel 21) of the sheath tube 20 through the second opening 16 on the control portion 10, and then cause the guidewire 50 to extend from the distal end 20A of the sheath tube 20 and enter the common bile duct or pancreatic duct through the intubation channel, thereby causing an associated instrument (for example, a balloon catheter, a net basket, an incision knife, a cell brush, a stent, a contrast catheter, a snare, etc.) to be inserted into the common bile duct or the pancreatic duct under the guidance of the guidewire 50 to perform the corresponding surgical operation. In some embodiments, the operator may inject the contrast agent into the channel (e.g., an injection channel) of the sheath tube 20 through the first opening 13 on the control portion 10, and then the contrast agent passes through the intubation channel and enters into the common bile duct or pancreatic duct for visualization.

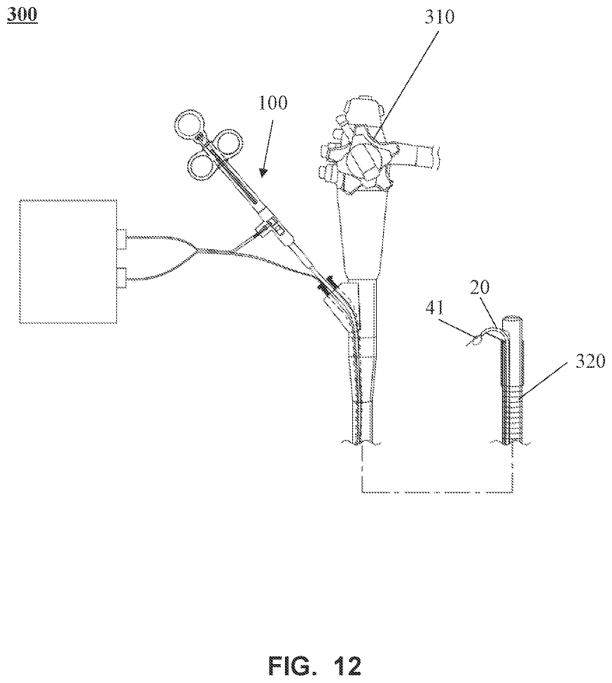

The embodiments of the present disclosure further provide an endoscope. The endoscope may include the endoscopic incision device 100 in any one of the embodiments of the present disclosure. FIG. 12 is a schematic diagram illustrating an endoscope according to some embodiments of the present disclosure. As shown in FIG. 12, an endoscope 300 may include the endoscopic incision device 100. Merely by way of example, the endoscope 300 may also include an operating handle 310 and an insertion portion 320, which may be inserted into a patient for imaging. In some embodiments, the insertion portion 320 may include an instrument channel, and the sheath tube 20 of the endoscopic incision device 100, as well as components (e.g., the pulling portion 40, the cutting wire 30, the guidewire 50, etc.) accommodated in the channel of the sheath tube 20 may be accommodated in the instrument channel. In some embodiments, the control portion 10 of the endoscopic incision device 100 may be provided independently relative to the operating handle 310 or may be integrated into the operating handle. During an intubation operation using the endoscopic device 300, the operator may insert the insertion portion 320 into the patient for imaging to locate an opening of the duodenal papilla, and by adjusting a bending angle and a bending direction of a distal end 20A of the sheath tube 20 of the endoscopic incision device 100, the distal end 20A of the sheath tube 20 may be made to enter the opening of the duodenal papilla, and then by using the protruding portion 41 to pull the duodenal papilla fold tissue to increase the width of the intubation channel, the difficulty of intubation is decreased and the success rate of intubation is increased.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

1. An endoscopic incision device, comprising:
a sheath tube, wherein the sheath tube has a proximal end and a distal end, the sheath tube is provided with a channel extending along an axial direction, and the channel is able to accommodate a pulling portion; wherein
the pulling portion includes a pulling wire and a protruding portion disposed at a distal end of the pulling wire, and the pulling wire moves axially within the channel to cause the protruding portion to switch between a first state and a second state;
wherein when the protruding portion is in the first state, the protruding portion is accommodated within the channel; and
when the protruding portion is in the second state, the protruding portion protrudes out of the distal end of the channel.

2. The endoscopic incision device of claim 1, wherein
when the protruding portion is in the second state, the protruding portion forms a joint portion with the distal end of the sheath tube, and a radial size of the joint portion is greater than a diameter of duodenal papilla; and
a radial size of the protruding portion is a first size, and a radial size from a most distal end of the protruding portion in a first radial direction to a most distal end of the sheath tube in a second radial direction is a second size; wherein
when the first size is greater than the second size, a radial size of the joint portion is the first size; and
when the second size is greater than the first size, the radial size of the joint portion is the second size.

3. The endoscopic incision device of claim 1, wherein
the radial size of the protruding portion in the second state is larger than the radial size of the protruding portion in the first state;
the radial size of the protruding portion in the first state is less than or equal to a radial size of the distal end of the sheath tube; and
the radial size of the protruding portion in the second state is greater than the radial size of the distal end of the sheath tube.

4. The endoscopic incision device of claim 3, wherein
the radial size of the protruding portion in the second state is within a range of 2 to 10 mm; and
the radial size of the distal end of the sheath tube is within a range of 1.8-2 mm.

5. The endoscopic incision device of claim 1, wherein the channel includes a first channel configured to accommodate a guidewire and a second channel configured to accommodate the pulling portion.

6. The endoscopic incision device of claim 5, wherein when switching from the first state to the second state, the protruding portion deflects away from an axis.

7. The endoscopic incision device of claim 6, wherein the second channel is provided eccentrically.

8. The endoscopic incision device of claim 5, wherein the distal end of the sheath tube is provided with a cutting wire configured to cut tissue, and the second channel collectively accommodates the pulling portion and the cutting wire.

9. The endoscopic incision device of claim 5, wherein the distal end of the sheath tube is provided with a cutting wire configured to cut tissue, and the channel further includes a third channel, the second channel accommodates the pulling portion and the third channel configured to accommodates the cutting wire.

10. The endoscopic incision device of claim 9, wherein the second channel or the third channel is an injection channel.

11. The endoscopic incision device of claim 9, wherein the channel further includes a fourth channel, the first channel accommodates the guidewire, the second channel accommodates the pulling portion, the third channel accommodates the cutting wire, and the fourth channel is an injection channel.

12. The endoscopic incision device of claim 5, wherein a cross-section of the first channel is a C-shaped cross-section, and the first channel includes a guidewire accommodating portion and a slit portion.

13. The endoscopic incision device of claim 1, wherein
the protruding portion is composed of one or more pulling lines to form a semi-closed or closed loop,
the protruding portion is composed of one or more pulling lines to form a semi-closed or closed mesh, or
the protruding portion is composed of one or more pulling lines to form a spiral.

14-15. (canceled)

16. The endoscopic incision device of claim 15, wherein a radial size of the protruding portion is approximately equal from the distal end to the proximal end, or gradually increases, or gradually decreases.

17. The endoscopic incision device of claim 1, further comprising a control portion, wherein the control portion includes a pulling handle, the pulling handle push-pulls axially or rotates to control the pulling wire to move axially within the channel, causing the protruding portion to switch between a first state and a second state.

18. The endoscopic incision device of claim 17, wherein when the protruding portion is in the second state, the pulling handle is further configured to control the pulling wire to move axially within the channel, causing the protruding portion to pull tissue.

19. The endoscopic incision device of claim 1, wherein the distal end of the pulling wire and a proximal end of the protruding portion are fixedly connected, or the distal end of the pulling wire and the proximal end of the protruding portion are detachably connected.

20. (canceled)

21. A method for operating the endoscopic incision device of claim 1, comprising:
controlling the pulling wire to move axially from the proximal end to the distal end of the sheath tube within the channel of the sheath tube, causing the protruding portion to switch from the first state to the second state; and
when the protruding portion is in the second state, controlling the pulling wire to move axially within the channel of the sheath tube, causing the protruding portion in the second state to pull duodenal papilla fold tissue within an intubation channel.

22. The method of claim 21, further comprising:
controlling the pulling wire to move axially from the distal end to the proximal end of the sheath tube within the channel of the sheath tube, causing the protruding portion to switch from the second state to the first state; and
when the protruding portion is in the first state, controlling a guidewire to extend from the distal end of the sheath tube and enter common bile duct or pancreatic duct through the intubation channel.

23. An endoscope, comprising the endoscopic incision device of claim 1.
